(19) United States
(12) Patent Application Publication
(10) Pub. No.: US 2024/0404645 A1
(43) Pub. Date: Dec. 5, 2024

(54) METHOD FOR OPERATING A CHEMICAL PRODUCTION SYSTEM

(71) Applicant: BASF SE, Ludwigshafen (DE)

(72) Inventors: Konstantin Schneider, Ludwigshafen (DE); Kathrin Wolter, Lampertheim (DE); Christina Koeppen, Ludwigshafen (DE); Miriam Mathea, Ludwigshafen (DE); Lars Meyer, Ludwigshafen (DE); Paul-Albert Anselm Schneide, Ludwigshafen (DE)

(21) Appl. No.: 18/695,424

(22) PCT Filed: Sep. 27, 2022
(86) PCT No.: PCT/EP2022/076746

§ 371 Date: Mar. 26, 2024

(30) Foreign Application Priority Data
Oct. 1, 2021 (EP) 21200568.0

(51) Int. Cl. G16C 20/10; G05B 19/406

(52) U.S. Cl. CPC G16C 20/10; G05B 19/406; G05B 2219/32287

(57) ABSTRACT

The disclosure concerns a computer-implemented method for monitoring and/or controlling a chemical and/or biological production process of an output product, wherein the output product is produced from one or more input product(s), the method comprising the steps of: providing spectral measurement data associated with characteristics of the input product and/or the output product, determining at least one performance indicator based on the spectral measurement data using a data-driven model, wherein the data-driven model is parameterized according to historical data comprising spectral measurement data associated with characteristics of the input product and/or the output product, wherein the performance indicator is a validation indicator indicating that spectral measurement data lies in a valid range, providing the performance indicator for monitoring and/or controlling the chemical and/or biological production process of the output product.

### TECHNICAL FIELD

The invention relates to a computer-implemented method for producing an intermediate product based on raw materials and a computer program.

### BACKGROUND ART

In current chemical environment different techniques are used to qualify chemicals along different stages in the value chain. Such techniques include state of the art analytics to analyze chemical compositions or to derive performance parameters. Classical use cases are laboratory analytics in research and development, quality control measurements in production lines or technical engineering services for customers.

The measurement techniques thus far used in such cases are highly diverse and do not produce comparable or reproducible results. This leads to a very diverse landscape with regard to measurement set up and information flows. Additionally, any data produced in such diverse environment is of limited use for big data applications. First hardware exists so materials can be characterized and compared across different measurement equipment at different points in time. The integration of such equipment is, however, in its infancy and more reliable data analytics are needed.

Hence there is a need to improve the integration of scalable measurement equipment for the characterization of material properties.

### SUMMARY

It is therefore desirable to provide methods and apparatuses which use reproducible spectra to improve process control in chemical production.

In one aspect a computer-implemented method for monitoring and/or controlling a chemical and/or biological production process of an output product is provided, wherein the output product is produced from one or more input product(s), the method comprising the steps of:
providing spectral measurement data associated with characteristics of the input product and/or the output product,
determining at least one performance indicator based on the spectral measurement data using a data-driven model, wherein the data-driven model is parameterized according to historical data comprising spectral measurement data associated with characteristics of the input product and/or the output product, wherein the performance indicator is a validation indicator indicating that spectral measurement data lies in a valid range, providing the performance indicator for monitoring and/or controlling the chemical and/or biological production process of the output product.

In another aspect a computer-implemented method for selecting a chemical and/or biological production facility or process is provided, the method comprising the steps of:
providing spectral measurement data associated with characteristics of one or more input product(s) and/or output product(s),
determine at least one performance indicator based on data driven model(s) associated with different production facilities or processes, wherein the data driven models are parametrized based on historical data associated with each production facility or process, wherein preferably each data-driven model may be parameterized according to historical data comprising spectral measurement data related to the input product(s) and/or output product(s) for one production facility or process and wherein the performance indicator is a validation indicator indicating that spectral measurement data lies in a valid range,
providing the performance indicator for selecting a chemical and/or biological production facility or process.

In another aspect a computer-implemented method for selecting a data driven model used in any of the methods disclosed herein, the method comprising the steps:
providing an input product identifier, an output product identifier, a production facility identifier and/or a production process identifier,
retrieving based on the input product identifier, the output product identifier, the production facility identifier and/or the production process identifier, a data-driven model for providing spectral measurement data in association with the provided input product identifier, the provided output product identifier, the provided production facility identifier and/or the provided production process identifier to the data driven model.

In another aspect a computer element with instructions is provided, which when executed by one or more computing apparatus(es) cause the one or more computing apparatus(es) to carry out the steps according to any of methods disclosed herein.

Validating output and/or input products based on spectral measurement data can enhance production to provide more reliable outputs. Additionally, process scale up is facilitated because influences of the scale factor onto the process performance (e.g. minor component levels) can be tracked. Controlling and/or monitoring production processes based on spectral measurement data can enhance reliability of production processes. Providing models to be used at different stages of the production process allows for flexible operation improving the overall production process chain.

An apparatus for monitoring and/or controlling a chemical and/or biological production process of an output product, for selecting a chemical and/or biological production facility or process and/or for selecting a data driven model, the apparatus comprising processing units with instructions for monitoring and/or controlling a chemical and/or biological production process of an output product, for selecting a chemical and/or biological production facility or process and/or for selecting a data driven model, which when executed by one or more computing apparatus(es) cause the one or more computing apparatus(es) to carry out the steps according to any of methods disclosed herein.

Use of a performance indicator as obtained by any one of the methods for monitoring and/or controlling a chemical and/or biological production process of an output product.

Spectral measurement data may comprise data generated by means of spectroscopical methods. Spectroscopical methods may comprise methods for qualitative analysis, quantitative analysis and/or structural analysis. Hence, spectral measurement data may comprise data suitable for determining qualitative information, quantitative information and/or structural information. Spectral measurement data may comprise numerical values, preferably numerical values in an array structure. Spectral measurement data may comprise spectra and/or data suitable for representing and/or obtaining spectra. A spectrum may be a distribution function of a physical quantity. Such physical quantities may be for example, wavelength, frequency, wavenumber, energy, any quantity derived from the exemplary quantities and/or the like. Spectra may be obtained by performing mathematical operations on spectral measurement data, such as derivation, integration, multiplication or the like. Examples for data suitable for obtaining spectra may be interferograms, derivatives, antiderivative and/or other kinds of data related to spectra. Spectrum may be obtained based on an interferogram, e.g. by performing a Fourier transform. This is known in the art. Interferogram may relate a measure for the intensity of light to a measure of the phase difference of at least a part of the light.

The performance indicator is a validation indicator indicating that spectral measurement data lies in a valid range or a time-dependent product performance indicator associated with performance characteristics of the output product. A validation indicator may used for validation in relation to the input product and/or the output product or for selection of the production facility or process and/or an input product. The time-dependent product performance may be used for monitoring and/or controlling the production facility or process, preferably in real-time during the production process.

In one embodiment the validation indicator may be a classifier indicating that the spectral measurement data associated with the input product and/or the output product lies in a valid or invalid range. Operation parameters of the production process may be determined and provided, if the input product is classified valid and/or if the output product is classified not valid.

In an example the validation indicator may be a classifier indicating that the spectral measurement data associated with the input product lies in a valid or invalid range. If the spectral measurement data does not lie in a valid range the input product may be classified invalid and the input product may be excluded from production. If the spectral measurement data does lie in a valid range the input product may be classified valid and associated operation parameters of the production process may be used.

In another example the validation indicator may be a classifier indicating that the spectral measurement data associated with the output product lies in a valid or invalid range. If the spectral measurement data does not lie in a valid range, the output product associated with the output product identifier is classified invalid. The operation parameters of the production process may be adjusted, if the output product is classified not valid.

In one embodiment the spectral measurement data includes one or more infrared spectra and/or one or more interferograms. Infrared spectra are particularly suitable to derive consistent measurement results across different equipment. Infrared spectra can be obtained from interferograms.

In one embodiment the historical data includes spectral measurement data provided from different measurement equipment, preferably from different measurement equipment of one measurement type. The historical data may include spectral measurement data provided from different measurement equipment that measures infrared spectra. Such spectra may be provided reproducibly across different analyzers by commercially available mid infrared analyzers.

In one embodiment historical data includes time-dependent spectral measurement data. The time-dependent spectral measurement data may be used to determine a time-dependent product performance indicator. The time-dependent product performance indicator associated with performance characteristics of the output product may be determined. This may allow for determination of time-dependent product performance, which may be used for monitoring and/or controlling the production facility or process, preferably in real-time during the production process.

In one example the spectral measurement data is time-dependent spectral measurement data related to characteristics of the production process of the output product The spectral measurement data may be provided for different points in time, preferably following a measurement protocol. At least one performance indicator for the production process may be determined based on the spectral measurement data using a data-driven model, wherein the data-driven model is parameterized according to historical data comprising time-dependent spectral measurement data related to the performance indicator. Based on the performance indicator one or more adjusted operation parameter(s) for the production process may be determined. In addition, an adjusted measurement protocol for providing spectral measurement data may be determined. The adjusted operation parameter(s) for monitoring and/or controlling the production process may be provided. In addition, the adjusted measurement protocol may be provided. In addition, further measurement of spectral measurement data may be triggered according to the adjusted measurement protocol.

In one embodiment the chemical and/or biological production process of the output product is monitored and/or controlled based on the performance indicator, wherein operation parameters for monitoring and/or controlling the chemical and/or biological production process are determined based on the performance indicator. The production process may be monitored and/or controlled according to the adjusted operation parameter(s).

In one embodiment the chemical and/or biological production process of the output product is monitored and/or controlled based on the performance indicator preferably associated with the input or output product. Operation parameters for monitoring and/or controlling the chemical and/or biological production process may be determined based on the performance indicator.

The performance indicator for monitoring and/or controlling the chemical and/or biological production process of the input and/or output product may be provided for the selected production facility or process. The chemical and/or biological production process of the output product may be monitored and/or controlled based on the performance indicator for the selected production facility or process. This may include operation parameters for monitoring and/or controlling the chemical and/or biological production process determined based on the performance indicator. For more than one input product more than one performance indicator may be determined.

In one embodiment, at least one value indicating the valid and/or invalid range may be provided, preferably by a costumer and/or supplier, and/or used for determining the at least one performance indicator. Value indicating the valid and/or invalid range may comprise a numerical value, preferably at least two numerical values. Value indicating the valid and/or invalid range may comprise an upper and/or lower limit for the performance indicator. Using the at least one value indicating the valid and/or invalid range may comprise comparing the performance indicator and the at least one value indicating the valid and/or invalid range. Based on this comparison, it may be determined that the performance indicator may be indicating that spectral measurement data lies in a valid or invalid range.

In one embodiment, performance indicator may comprise a numerical value, a validation status such as valid or invalid and/or may be a physical quantity and/or chemical quantity and/or biological quantity. For example, performance indicator may relate to an absolute and/or relative amount of a chemical compound, an absolute and/or relative amount of an impurity, a physical quantity associated with a compound or a mixture of compounds. Absolute and/or relative amount may be for example a mass or volume concentration, a density or the like. Examples for a physical quantities associated with a compound or a mixture of compounds may comprise solubility, steam point, electrical conductivity, viscosity or the like.

In one embodiment, the input product may be excluded from production and/or associated operation parameters may be adjusted if the analytical measurement data does not lie in a valid range and/or the operation parameters associated with the input product may be used if the analytical measurement data lies in a valid range.

### SHORT DESCRIPTION OF THE FIGURES

Further optional features and embodiments will be disclosed in more detail in the subsequent description of embodiments. The optional features may be realized in an isolated fashion as well as in any feasible combination, as the skilled person will realize. The scope of the invention is not restricted by the preferred embodiments. The embodiments are schematically depicted in the Figures. Therein, identical reference numbers in these Figures refer to identical or functionally comparable elements.

FIG. 1 shows a distributed chemical production system in which the method(s) described herein may be implemented.

FIG. 2 shows a flow chart of a computer-implemented method for operating a chemical production system 100 based on data-driven models relating to spectral measurement data.

FIG. 3 shows an example of flow chart of a computer-implemented method for operating a chemical production system 100 by validating an input product based on data-driven models relating to spectral measurement data.

FIG. 4 shows an example of an IR spectrum.

FIG. 5 shows an example of a flow chart of a computer-implemented method for operating a chemical production system 100 by selecting a production facility or process based on data-driven models relating to spectral measurement data.

FIG. 6 shows an example of flow chart of a computer-implemented method for operating a chemical production system 100 by validating the output product based on data-driven models relating to spectral measurement data.

FIG. 7 shows an example for validating a recipe formulation.

FIG. 8 shows an example of flow chart of a computer-implemented method for operating a chemical production system 100 by monitoring and/or controlling the production process based on data-driven models relating to spectral measurement data.

FIG. 9 shows an example for monitoring and/or controlling a fermentation process.

FIG. 10 shows an example for monitoring and/or controlling a polymerization process.

FIG. 11 shows an example for monitoring and/or controlling bacteria cells in a fermentation process.

FIG. 12 shows an example for monitoring and/or controlling a chemical and/or biological production process of an output product.

### DETAILED DESCRIPTION OF THE EMBODIMENTS

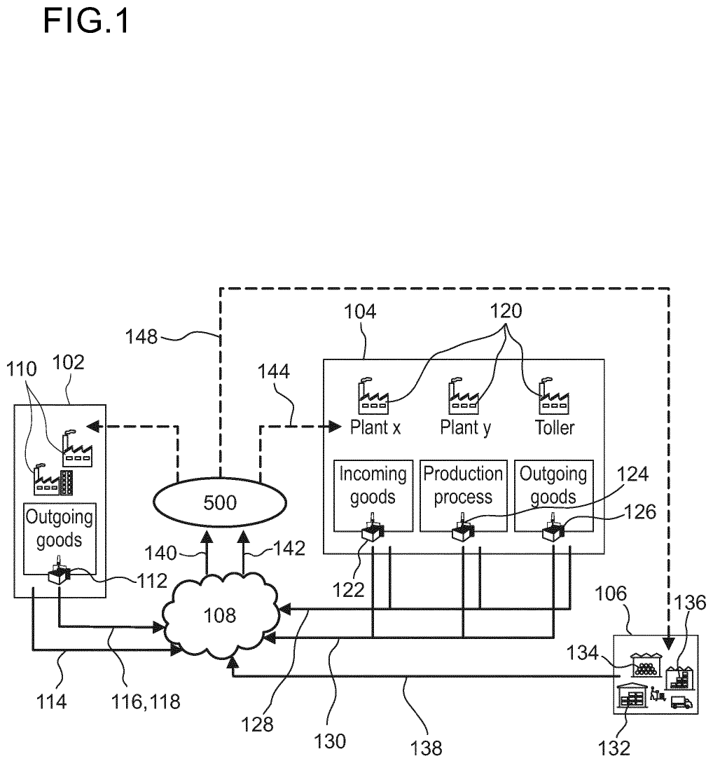

FIG. 1 shows a distributed chemical production system 100 in which the method(s) described herein may be implemented.

The distributed chemical production system 100 comprises an input product system 102, a production system 104 and a output product system 106. The input product system 102, the production system 104 and the output product system 106 are communicatively coupled with one another by means of a computing environment 108 such as a cloud environment. The computing environment 108 may comprise at least one computing apparatus and associated databases 500 for carrying out the method(s) described herein. Interfaces between the system components allow for data exchange between the computing environment 108, the input product system 102, the production system 104 and the output product system 106.

The input product system 102 may comprise computing devices associated with one or more production facilities 110. The production facilities 110 may be configured to produce at least one input product. The productions facilities 110 may include one or more measurement stations 112 configured to perform spectral measurements. Such spectral measurements may include spectral measurements of precursor products or input products. The input product system 102 may be configured to provide input product identifiers and associated spectral measurement data to the computing environment 108. For each input product identifier further metadata associated with the input product may be provided. Such metadata may include additional measurement data related to the input product, performance specifications of the input product, availability of the input product or the like. The input product identifier, associated spectral measurement data and optionally further metadata may be provided to the computing environment 108.

The production system 104 may comprise computing devices associated with one or more production facilities 120. The production facilities 120 may be independent of one another or may be interconnected e.g. in a Verbund system. The production facilities 120 may be configured to produce at least one output product. The productions facilities 102 may include one or more measurement stations 122, 124, 126 configured to perform spectral measurements. Such spectral measurements may include spectral measurements of input products and/or output products. The production measurement stations 122, 124, 126 may be communicatively coupled to the computing environment 108. Spectral measurement data may be provided to the computing environment 108. Further, measurement data collected during the production processes may be provided to the computing environment 108.

The output product system 106 may include further processing systems such as a client device 132 configured to provide output product performance characteristics or to trigger production of the output product, a computing apparatus 134 configured to trigger further processing of the output product, a measurement station 136 configured to provide spectral measurement data of the output product or any product manufactured from the output product. The output product system 106 may be communicatively coupled to the computing environment 108. The output product system 106 may be configured to provide data related to the output product to the computing environment 108.

The computing environment 108 may be configured to process spectral measurement data 140 and associated models 500 or data 140, 142 from the input product system 102, the production system 104 and/or the output product system 106. Such processing may include the generation, selection and/or use of data-driven models 500. The computing environment 108 may be configured to provide data 144, 148, 150 to the input product system 102, the production system 104 or the output product system 106. Different embodiments relating to the processing of spectral measurement data 116, 118, 126, 130, 138 and associated models 500 or data 140, 142 are described in a non-limiting manner by way of the following example flow charts.

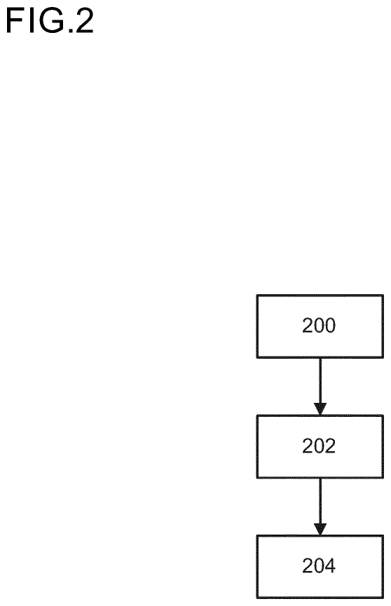

FIG. 2 shows a flow chart of a computer-implemented method for operating a chemical production based on data-driven models relating to spectral measurement data.

In a first step 200 an input product identifier, an output product identifier, a production facility identifier and/or a production process identifier may be provided. Additionally, metadata relating to spectral measurement data, performance data and/or operation data may be provided. Metadata may include spectral measurement data relating to the input product identifier, the output product identifier, the production facility identifier and/or the production process identifier.

In a second step 202, based on the input product identifier, the output product identifier, the production facility identifier and/or the production process identifier, a data-driven model 500 may be selected and/or retrieved from a data base. Such selection and/or retrieval may be based on metadata associated with the data-driven model. Metadata may correlate to the provided input product identifier, the provided output product identifier, the provided production facility identifier and/or the provided production process identifier. The data-driven models may be trained based on spectral measurement data of input products or output products as produced in a production facility or a production process 110, 120. The data-driven models may be associated with the provided input product identifier, the provided output product identifier, the provided production facility identifier and/or the provided production process identifier.

In a third step 204 the spectral measurement data provided in association with the provided input product identifier, the provided output product identifier, the provided production facility identifier and/or the provided production process identifier may be provided to the data driven model. By using the data-driven model a validation indicator in relation to the input product and/or the output product may be determined. By using the data-driven model a production facility or process may be selected. By using the data-driven model a production process may be monitored and/or controlled.

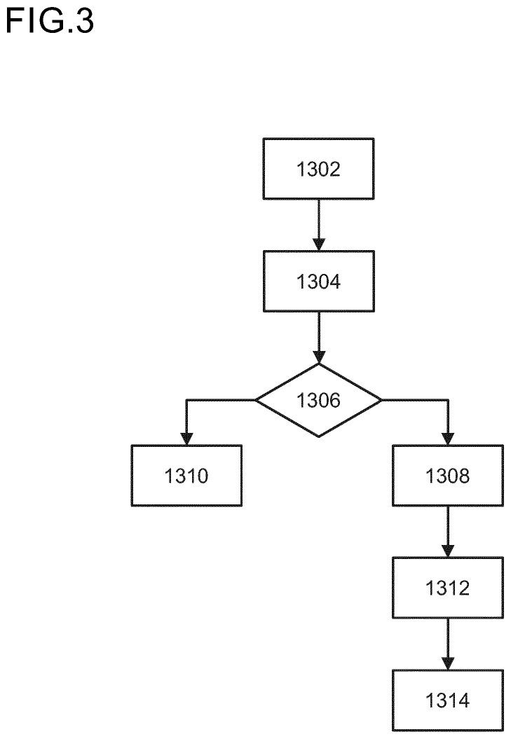

FIG. 3 shows an example of flow chart of a computer-implemented method for operating a chemical and/or biological production by validating an input product based on data-driven models relating to spectral measurement data. In this example outlier detection is used to suitably run the production process based on the input product to be used in the production process or production facility.

### Outlier Detection Input Product

In a first step 1302, spectral measurement data associated with characteristics of one or more input product(s) is provided. The spectral measurement data may include one or more IR, e.g. MIR or NIR, spectra. The spectral measurement data may signify transmission or absorption behavior in a wavelength, wavenumber or frequency range. The spectral measurement data may signify the chemical composition of the input material. The spectral measurement data may signify a chemical composition and relative amounts of individual chemical components of the chemical composition.

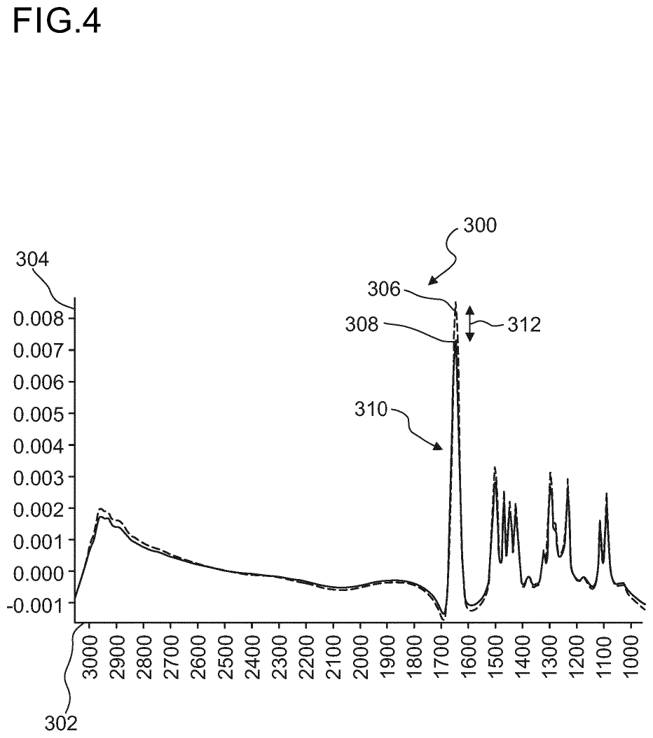

FIG. 4 shows an example of an IR spectrum 300 as used in the method(s) described herein. The graph 300 shows the wavenumber in nanometers on the x-axis 302 and absorption or transmittance on the y-axis 304. Two exemplary spectra 306, 308 are shown. As indicated by arrow 310, the peak of the IR absorption or transmission bands within the spectra 306, 308 provide characteristic information regarding the chemical composition of the input product. The position of the peak signifies the component or compound of a mixture. The intensity and shape of the peak signify the concentration or relative amount of the component or compound in the mixture. As is further indicated by arrow 312, for a given compound or component, the intensity of an absorption or transmission signal is proportional to the relative amount or concentration of this compound or component in a mixture of different compounds according to Beer's law.

IR spectrum 300 may be generated with an IR spectrometer. Different measurement setups can be realized depending on the spectrometer used. Dispersive spectrometers may generate a spectrum by measuring absorption, transmission and/or reflectance in relation to the corresponding measure for the energy of the incident light by scanning over a range. Another approach may include measuring absorption, transmission and/or reflectance of light with a variety of different measure for the energy of light, thus, generating an interferogram. The interferogram may be Fourier transformed into an IR spectrum 300.

In a second step 1304, a validation indicator is determined based on the spectral measurement data using a data-driven model, wherein the data-driven model is parameterized according to historical data comprising spectral measurement data related to the input product. For more than one input product more than one validation indicator may be determined. In such case the input products may differ in details of the chemical composition. Input products e.g. from different suppliers may include different impurities or fractions of chemical components making up the mixture.

The historical data may include spectral measurement data provided from different measurement equipment, preferably from different measurement equipment of one measurement type. The historical data may include spectral measurement data provided from different measurement equipment that measures infrared spectra. Such spectra may be provided reproducibly across different analyzers by commercially available MIR analyzers.

The historical data may include spectral measurement data associated with a desired performance of the output product. The historical data may include spectra associated with input product(s) that provided a desired performance of the output product in previous production runs. The historical data may include normalized spectra to achieve comparability between the spectra within the training data set. Normalization may be based on the amplitudes with respect to a reference peak or on the amplitudes with respect to the dominating component. Another option may be taking first or second derivatives of the spectra. Another option includes correction of the spectra with respect to equipment characteristics or environmental characteristics such as humidity or CO₂.

The data driven model may be a partial least square model or a model based on principal component analysis. In another embodiment the model may be based on a point wise median determination with confidence interval. In yet another embodiment the model may be based on clustering methods using a similarity measure.

In a third step 1306, it is determined if the validation indicator lies in a valid range. The validation indicator may be a classifier indicating that the spectral measurement data associated with the input product lies in a valid range.

If the spectral measurement data lies in a valid range, the input product associated with the input product identifier may be classified valid in a fourth step 1308. Valid in this context means that the input product may be provided to the chemical and/or biological production process to produce an output product with desired performance.

The validation indicator may be a classifier indicating that the spectral measurement data associated with the input product does not lie in a valid range. If the spectral measurement data does not lie in a valid range the input product associated with the input product identifier is classified invalid in a fifth step 1310. Invalid in this context means that the input product may not be provided to the chemical and/or biological production process to produce an output product with desired performance. This may include that there is a higher risk of producing 2nd class quality (example food vs. feed grade).

In a sixth step 1312, the validation indicator associated with the input product is provided for monitoring and/or controlling the chemical and/or biological production process of the output product. This may include operation parameters for monitoring and/or controlling the chemical and/or biological production process determined based on the validation identifier. The operation parameters of the production process may be adjusted, if the output product is classified valid.

In a seventh step 1314 the chemical and/or biological production process of the output product is monitored and/or controlled based on the validation indicator associated with the input product. This may include that operation parameters for monitoring and/or controlling the chemical and/or biological production process are determined based on the validation identifier. One example may include monitoring and/or controlling a feed unit of the production process to control and/or monitor the input product feed to the production process. This way the feed including feed rates, temperatures or the like under suitable conditions for the input product can be ensured.

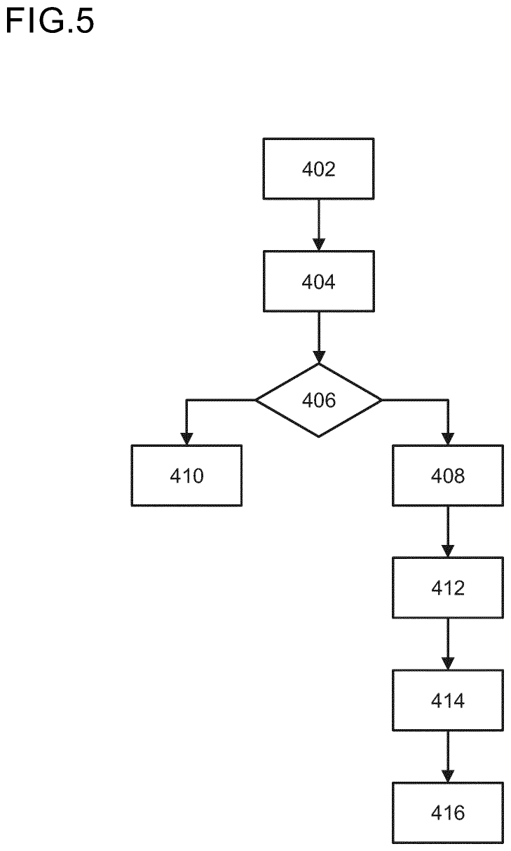

FIG. 5 shows an example of a flow chart of a computer-implemented method for operating a chemical and/or biological production by selecting a production facility or production process based on data-driven models relating to spectral measurement data. In this example outlier detection is used to match the suitable production facility or production process.

Outlier Detection Input Product Combined with Production Facility Selection

In a first step 402, spectral measurement data associated with characteristics of one or more input product(s) is provided. The spectral measurement data may signify the chemical composition as described in the context of FIGS. 4 and 5.

In a second step 404, a validation indicator is determined based on the spectral measurement data using data-driven models associated with different production facilities or processes, each data-driven model is parameterized according to historical data comprising spectral measurement data related to the input product for one production facility or process.

Here data-driven models may be associated with different production facilities or processes. Each data driven model may be parametrized based on historical data associated with one production facility or process. The data driven models may be production facility or process specific. For more than one input product more than one validation indicator may be determined using one data-driven model associated with one production facility or process. In such case the input products may differ in details of the chemical composition. Input products e.g. from different suppliers may include different impurities or fractions of chemical components making up the mixture.

The historical data may include spectral measurement data provided from different measurement equipment, preferably from different measurement equipment of one measurement type. The historical data may include spectral measurement data provided from different measurement equipment that measures infrared spectra. Such spectra may be provided reproducibly across different analyzers by commercially available MIR analyzers.

The historical data may include spectral measurement data associated with a desired performance of the output product. The historical data may include spectra associated with input product(s) that provided a desired performance of the output product in previous production runs. The historical data may include normalized spectra to achieve comparability between the spectra within the training data set. Normalization may be based on the amplitudes with respect to a reference peak or on the amplitudes with respect to the dominating component.

The production facility or process specific data driven model may be a partial least square model or a model based on principal component analysis. In another embodiment the model may be based on a point wise median determination with confidence interval. In yet another embodiment the model may be based clustering methods using a similarity measure.

In a third step 406, it is determined if the validation indicator lies in a valid range. The validation indicator may be a classifier indicating that the spectral measurement data associated with the input product lies in a valid range for a production facility or process.

If the spectral measurement data lies in a valid range for a production facility or process, the input product associated with the input product identifier may be classified valid for such production facility or process in a fourth step 408. Valid in this context means that the input product be provided to the chemical and/or biological production process to produce an output product with desired performance for such production facility.

The validation indicator may be a classifier indicating that the spectral measurement data associated with the input product does not lie in a valid range a production facility in a fifth step 410. If the spectral measurement data does not lie in a valid range for a production facility or process, the input product associated with the input product identifier may be classified invalid for such production facility or process. Invalid in this context means that the input product may not be provided to the chemical and/or biological production process to produce an output product with desired performance for such production facility.

In a sixth step 412, one production facility or process may be selected. If only one production facility is associated with a validation indicator signifying a valid input product, the selection step may be redundant. If more than one production facility is associated with a validation indicator signifying a valid input product, one production facility or process may be selected. In one embodiment any production facility or process may be selected. In such embodiment further selection criteria such as capacity, production schedule or the like may be used. In another embodiment the validation indicators for different production facilities or processes may by ranked according to a validation distance, e.g. from a center of a cluster. The production facility or process with the lowest validation distance may be selected.

In a seventh step 414, the validation indicator for monitoring and/or controlling the chemical and/or biological production process of the output product is provided for the selected production facility or process.

In a eighth 416 step, the chemical and/or biological production process of the output product is monitored and/or controlled based on the validation indicator associated with the selected production facility or process. This may include operation parameters for monitoring and/or controlling the chemical and/or biological production process determined based on the validation identifier. This may include monitoring and/or controlling a feed unit of the production process of the selected production facility or process to control and/or monitor the input product feed to the production process. This way process conditions such as temperature, flow, pH as adaption to the input product(s) may be monitored and/or controlled.

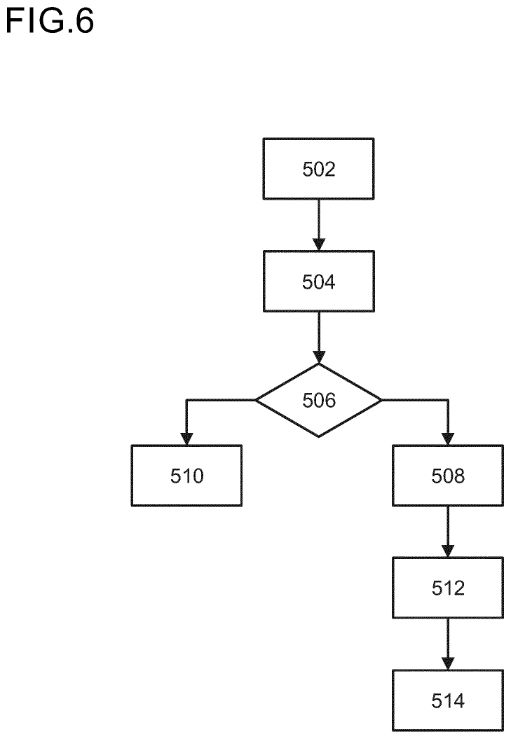

FIG. 6 shows an example of flow chart of a computer-implemented method for operating a chemical and/or biological production by validating the output product based on data-driven models relating to spectral measurement data. In this example outlier detection is used in relation to the output product for recipe or quality validation.

### Outlier Detection (Recipe Check, Quality Check, Contamination Check)

In a first step 502, spectral measurement data associated with characteristics of the output product of the production process is provided. The spectral measurement data may signify the chemical composition as described in the context of FIGS. 4 and 5.

In a second step 504, a validation indicator is determined based on the spectral measurement data using a data-driven model, wherein the data-driven model is parameterized according to historical data comprising spectral measurement data related to the output product.

The historical data may include spectral measurement data provided from different measurement equipment, preferably from different measurement equipment of one measurement type. The historical data may include spectral measurement data provided from different measurement equipment that measures infrared spectra. Such spectra may be provided reproducibly across different analyzers by commercially available MIR analyzers.

The historical data may include spectral measurement data associated with a performance of the output product. The performance may relate to the concentration of components or the concentration of impurities. The historical data may include spectra associated with the performance of the output product in previous production runs. The historical data may include normalized spectra to achieve comparability between the spectra within the training data set. Normalization may be based on the amplitudes with respect to a reference peak or on the amplitudes with respect to the dominating component.

The data driven model may be a partial least square model or a model based on principal component analysis. In another embodiment the model may be based on a point wise median determination with confidence interval. In yet another embodiment the model may be based clustering methods using a similarity measure.

In a third step 506, it is determined if the validation indicator lies in a valid range. The validation indicator may be a classifier indicating that the spectral measurement data associated with the output product lies in a valid range. If the spectral measurement data lies in a valid range, the output product associated with the output product identifier may be classified valid in step 510. Valid in this context means that the output product produced in the chemical and/or biological production process adheres to the desired performance.

The validation indicator may be a classifier indicating that the spectral measurement data associated with the output product does not lie in a valid range. If the spectral measurement data does not lie in a valid range the output product associated with the output product identifier is classified invalid in fifth step 508 Invalid in this context means that the output product produced in the chemical and/or biological production process does not adheres to the desired performance.

In a sixth step 512, the validation indicator for monitoring and/or controlling the chemical and/or biological production process of the output product is provided. This may include operation parameters for monitoring and/or controlling the chemical and/or biological production process determined based on the validation indicator. For instance, the operation parameters of the production process may be adjusted, if the output product is classified not valid.

In a seventh step 514 the chemical and/or biological production process is monitored and/or controlled based on the validation indicator. This may include monitoring and/or controlling a feed unit of the production process to control and/or monitor the recipe formulation to the production process. This way the feed of suitable recipe formulation can be ensured.

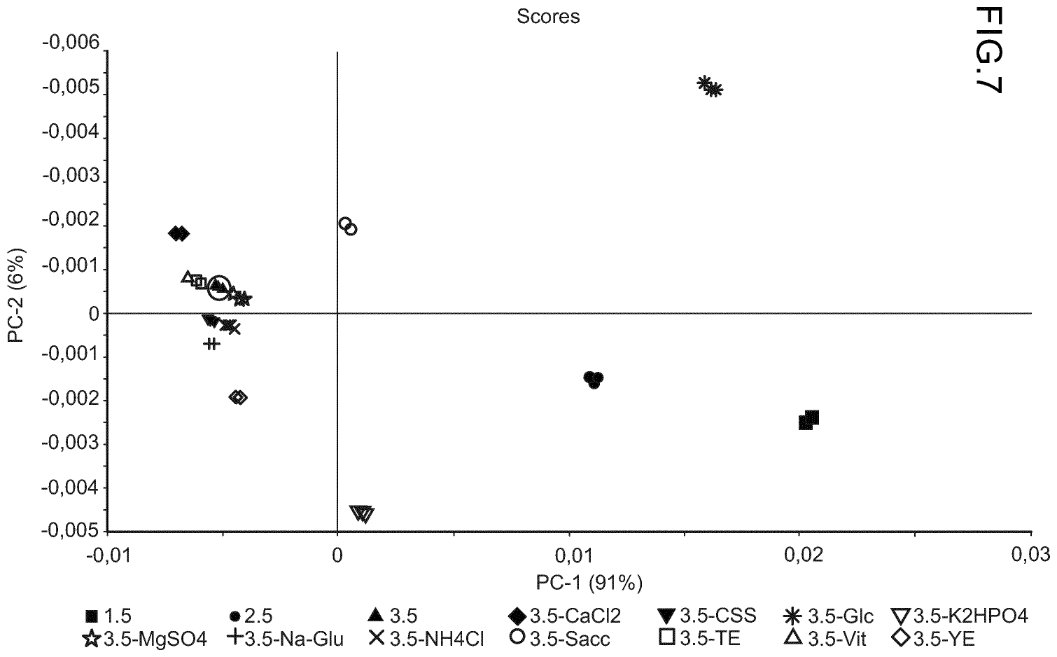

FIG. 7 shows an example for validating a recipe formulation.

Shown are the results from principal components analysis plotting the scores for principal component 1 PC1 vs principal component 2 PCS2. The spectral scores are plotted for different batches. The spectral scores are used to assess recipe quality on a qualitative and quantitative level.

The statistical analysis shows any deviation from the proper recipe (circle) either by lacking different components which are denoted below the graph or by incorrect amounts of e.g. sugar (red 2.5×, blue 1.5× instead of green 3.5×). Lacking components can be identified qualitatively due to their characteristic attribution to the spectral fingerprint. In this example spectral and statistical analysis prevents fail batches due to false recipe formulation. The corresponding model can be directly linked to the process control system of the production process to trigger manual or model-based actions thus increasing capacity by decreasing the number of failed batches.

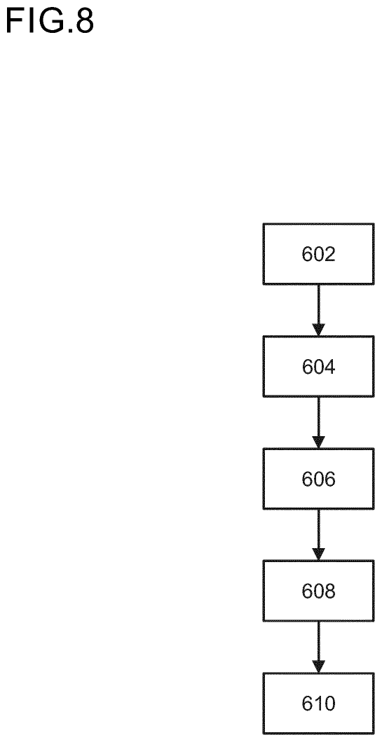

FIG. 8 shows an example of flow chart of a computer-implemented method for operating a chemical and or biological production by monitoring and/or controlling the production process based on data-driven models relating to spectral measurement data.

### Time Dependent Pca and Performance Prediction

In a first step 602, time-dependent spectral measurement data related to characteristics of the production process of the output product is provided. The spectral measurement data may be provided for different points in time, preferably following a measurement protocol. The spectral measurement data may signify the chemical composition as described in the context of FIGS. 4 and 5.

In a second step 604, at least one performance indicator for the production process is determined based on the spectral measurement data using a data-driven model, wherein the data-driven model is parameterized according to historical data comprising time-dependent spectral measurement data related to the performance indicator.

The historical data may include time-dependent spectral measurement data provided from different measurement equipment, preferably from different measurement equipment of one measurement type. The historical data may include time-dependent spectral measurement data provided from different measurement equipment that measures infrared spectra. Such spectra may be provided reproducibly across different analyzers by commercially available MIR analyzers.

The historical data may include time-dependent spectral measurement data related to the performance indicator. The performance indicator may relate to the concentration of one or more components. The historical data may include time-dependent spectra related to the performance indicator in previous production runs. The historical data may include normalized spectra to achieve comparability between the spectra within the training data set. Normalization may be based on the amplitudes with respect to a reference peak or on the amplitudes with respect to the dominating component.

The data driven model may be a partial least square model or a model based on principal component analysis. In another embodiment the model may be based on a point wise median determination with confidence interval. In yet another embodiment the model may be based on clustering methods using a similarity measure.

In a third step 606, based on the performance indicator one or more adjusted operation parameter(s) for the production process are determined. In addition, an adjusted measurement protocol for providing spectral measurement data may be determined.

In a fourth step 608, the adjusted operation parameter(s) for monitoring and/or controlling the production process are provided. In addition, the adjusted measurement protocol may be provided.

In a fifth step 610, the production process may be monitored and/or controlled according to the adjusted operation parameter(s). In addition, measurement of spectral measurement data may be triggered according to the adjusted measurement protocol may be provided.

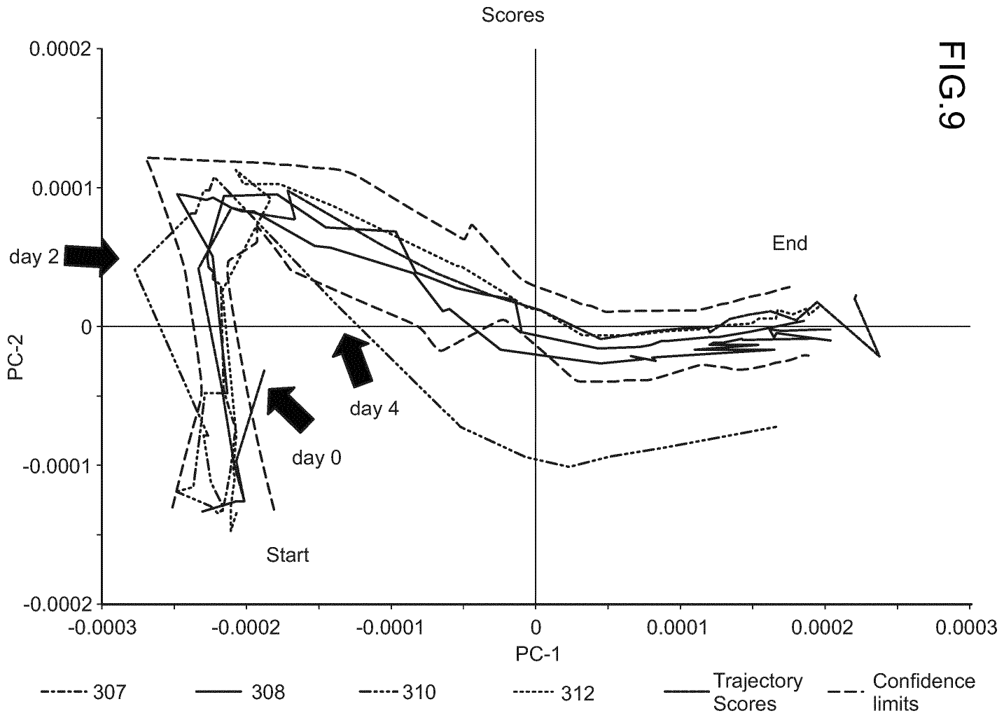

FIG. 9 shows an example for monitoring and/or controlling a fermentation process.

Shown are the results from a time-dependent principal analysis plotting time dependent scores for principal component 1 PC1 vs principal component 2 PCS2. Spectral measurement data is used for batch modelling. For different time points PC1 and PC2 were extracted from spectral measurement data as measured in previous production runs. From the analysis of historical data for different runs, that lead to a desired output product, a median and a confidence interval may be determined. Fermentation processes within range behavior lie within the confidence interval. The confidence interval represents the batch variation of the microbial fermentation process. For model derivation a multivariate analysis of batch process data was conducted to quantify the acceptable variability of the process variables during normal processing conditions as a function of the percent of batch completion or time. The resulting model can be used on new batch process data to identify measurements which indicate abnormal processing behavior.

Blue, green and light blue process were in range. The red fermentation was contaminated and shows a distinct deviation from in range runs. Thus, the method can be used to monitor a contamination event throughout the process and gives a faster response compared to traditional plate based contamination assays. The model can be directly linked to a process control system to manually or model-based trigger actions e.g. inactivation and disposal of the process fluid. Further the model can directly feed into the process planning and scheduling systems to e.g. adjust cycle times and/or automatic ordering/updating raw materials.

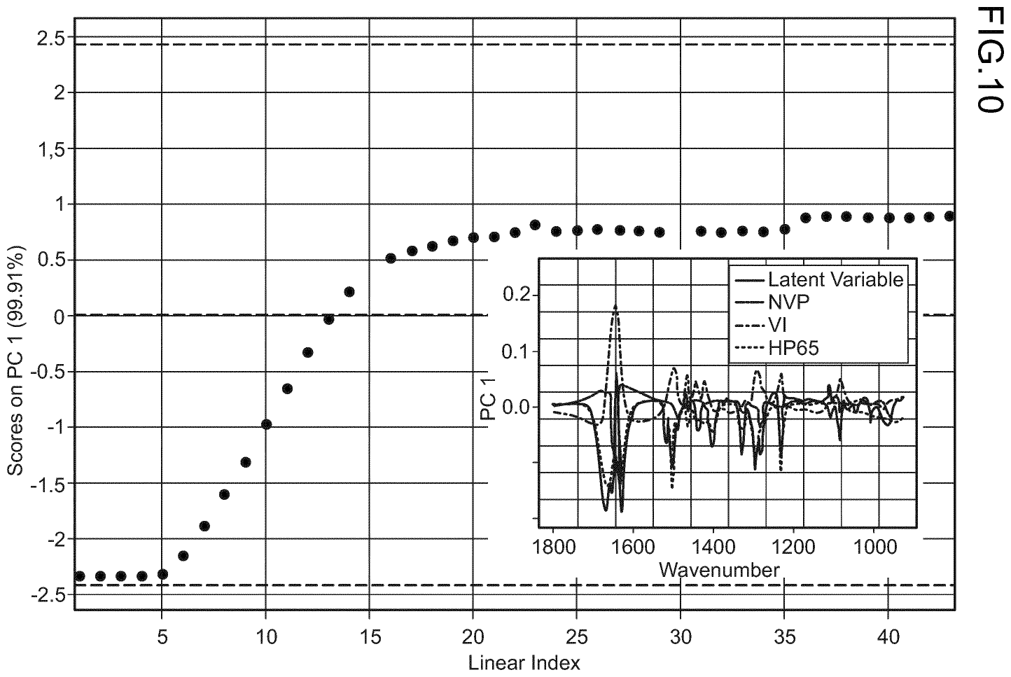

FIG. 10 shows an example for monitoring and/or controlling a polymerization process.

A polymerization reaction can be monitored using the spectral measurement data in combination with multivariate data analysis. The information encoded in the spectral measurement data enable different levels of analysis:
1. An untargeted high-level analysis comparing the process trajectories of different batch runs against a reference batch and detect anomalies in the evolving batch
2. A targeted low-level analysis of key performance/product quality indicators which maybe embedded as into a process control system.

The example of FIG. 10 shows the first principle component extracted from a time series of spectral measurements gathered during the polymerization plotted against the time. The first principal component is shown and describes >99.9% of the batch variance. The principal component shows the decrease of monomers and the increase of the polymer, as can be concluded from comparing the spectral loadings with reference spectra of the individual components. A PLS model can predict the residual monomer concentrations from the spectral measurement data. The post polymerization time for the observed reaction has been defined very long to guarantee low levels of residual monomer. Nevertheless, it is obvious, that in the end phase of the batch there is almost no change in the residual monomer level. Based on the spectral measurement data, a very fast analysis of the residual monomer concentration could be performed to check, if the post polymerization time could be shortened for the current batch. With other techniques than IR measurements, measurement can take several hours and therefore does not provide sufficient time resolution.

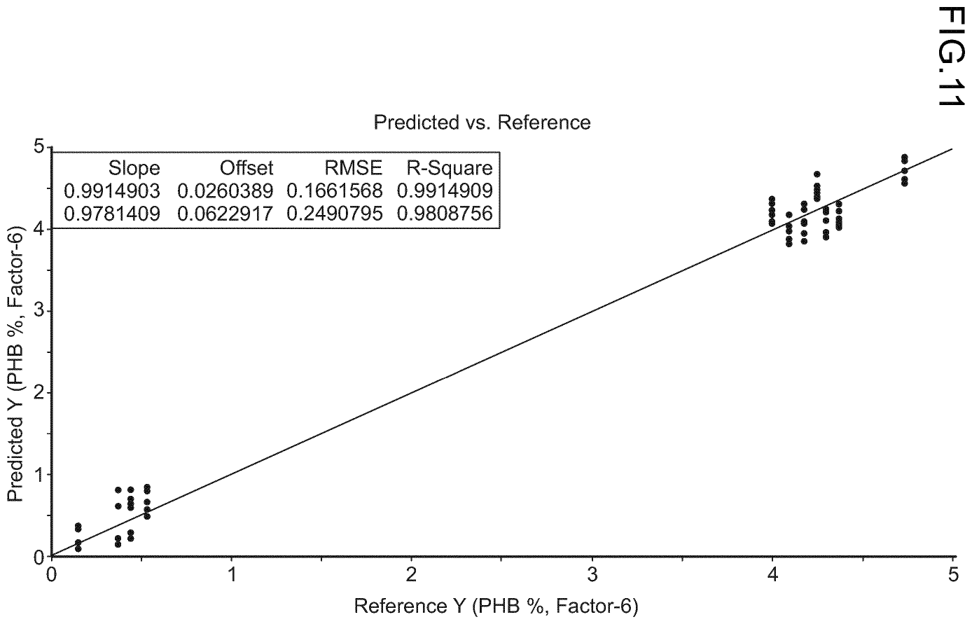

FIG. 11 shows an example for monitoring and/or controlling bacteria cells in a fermentation process.

Based on spectral data quality attributes of the insoluble product (bacterial cells) can be analyzed indirectly from the cell free matrix of the process liquid. The figure shows the model-based prediction of the quality attribute versus the measurement of the corresponding attribute. The model can directly feed into the process control system to estimate endpoints of the fermentation process based on the predicted quality attribute thus reducing cycle time and increasing plant capacity. Also direct feedback of the model-based predictions to process planning and scheduling systems allows to automatically reduce cycle times.

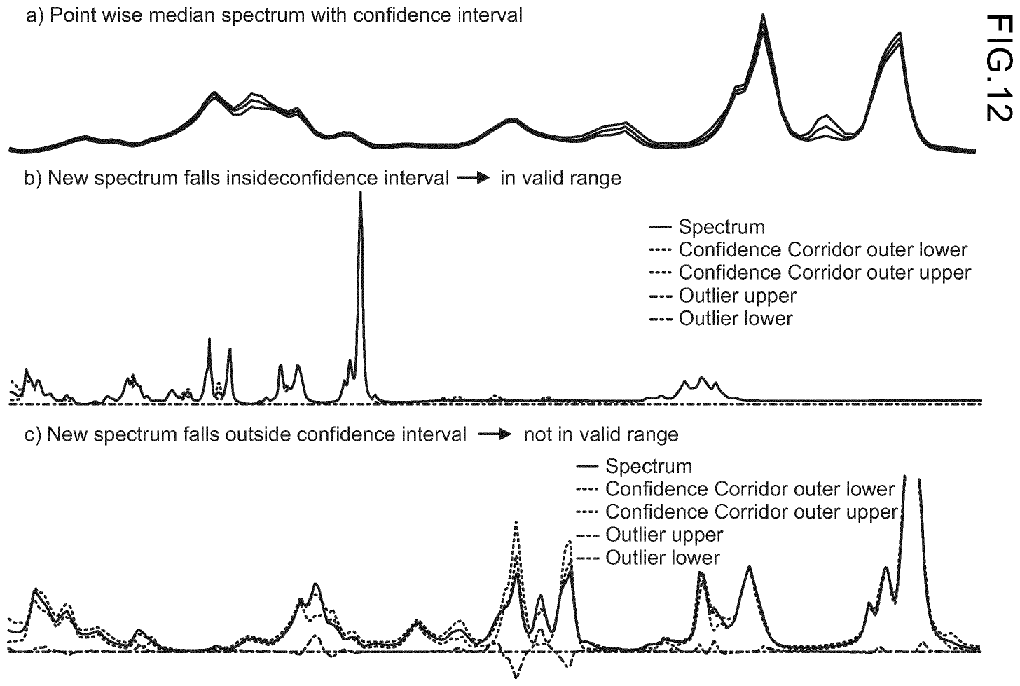

FIG. 12 shows an example for monitoring and/or controlling a chemical and/or biological production process of an output product.

Spectral measurement data associated with characteristics of the input product and/or the output product may be generated. Spectral measurement data associated with characteristics of the input product and/or the output product may be provided to a model. Model may determine at least one performance indicator based on the spectral measurement data. The model may be parameterized according to historical data comprising spectral measurement data associated with characteristics of the input product and/or the output product. Historical data may comprise spectral measurement data generated at an earlier point in time than spectral measurement data used for determining at least one performance indicator with the model. Model may be based on a point wise median determination with confidence interval as it can be seen in FIG. 12a. Spectral measurement data may lie within the confidence interval. Consequently, the performance indicator determined based on the spectral measurement data may indicate that spectral measurement data may lie in a valid range. In FIG. 12b an example for spectral measurement data lying in a valid range can be seen. The spectral measurement data may be associated with a low purity input and/or output product. Model may be trained based on spectral measurement data associated with a low purity input and/or output product. In FIG. 12c an example for spectral measurement data lying in an invalid range can be seen. Spectral measurement data and/or part of spectral measurement data lying in an invalid range may be referred to as outlier. Outliers may arise from the input product, recipe, quality and/or contamination of the product. Any of these can be linked to product performance indicators. Spectral measurement data may be associated with a high purity input and/or output product. Spectral measurement data shown in FIG. 12c may correspond to an outlier due to high quality. High quality may be achieved with a different recipe than a recipe that may be used for the production of input and/or output product associated with spectral measurement data in FIG. 12b.

1. A computer-implemented method for monitoring and/or controlling a chemical and/or biological production process of an output product, wherein the output product is produced from one or more input product(s), the method comprising:
providing spectral measurement data associated with characteristics of the input product and/or the output product,
determining at least one performance indicator based on the spectral measurement data using a data-driven model, wherein the data-driven model is parameterized according to historical data comprising spectral measurement data associated with characteristics of the input product and/or the output product, wherein the at least one performance indicator is a validation indicator indicating that spectral measurement data lies in a valid range,
providing the at least one performance indicator for monitoring and/or controlling the chemical and/or biological production process of the output product.

2. The method of claim 1, wherein the validation indicator is a classifier indicating that the spectral measurement data associated with the input product and/or the output product lies in a valid or an invalid range.

3. The method of claim 1, wherein at least one value indicating the valid and/or invalid range is provided and/or used for determining the at least one performance indicator.

4. The method of claim 1, wherein an operation parameter of the production process is provided, if the input product is classified valid and/or if the output product is classified not valid.

5. The method of claim 1, wherein the spectral measurement data includes one or more infrared spectra and/or one or more interferograms.

6. The method of claim 1, wherein the historical data includes spectral measurement data provided from different measurement equipment.

7. The method of claim 1, wherein the historical data includes time-dependent spectral measurement data.

8. The method of claim 7, wherein time-dependent spectral measurement data is used to determine a time-dependent product performance indicator.

9. The method of claim 7, wherein the time-dependent product performance indicator associated with performance characteristics of the output product is determined and used for monitoring and/or controlling the production facility or process in real-time during the production process.

10. The method of claim 1, wherein the chemical and/or biological production process of the output product is monitored and/or controlled based on the performance indicator, wherein operation parameters for monitoring and/or controlling the chemical and/or biological production process are determined based on the performance indicator.

11. The method of claim 1, wherein the input product is excluded from production and/or associated operation parameters are adjusted if the analytical measurement data does not lie in a valid range and/or the operation parameters associated with the input product are used if the analytical measurement data lies in a valid range.

12. A computer-implemented method for selecting a chemical and/or biological production facility or process, the method comprising:
providing spectral measurement data associated with characteristics of one or more input product(s) and/or the output product(s),
determine at least one performance indicator based on data driven model(s) associated with different production facilities or processes, wherein the data driven models are parametrized based on historical data associated with each production facility or process and wherein the performance indicator is a validation indicator indicating that spectral measurement data lies in a valid range,
providing the performance indicator for selecting a chemical and/or biological production facility or process.

13. A computer-implemented method for selecting a data driven model used in the method of claim 1, the method comprising:
providing an input product identifier, an output product identifier, a production facility identifier and/or a production process identifier,
retrieving based on the input product identifier, the output product identifier, the production facility identifier and/or the production process identifier, a data-driven model for providing spectral measurement data in association with the provided input product identifier, the provided output product identifier, the provided production facility identifier and/or the provided production process identifier to the data driven model.

14. A computer element with instructions, which when executed by one or more computing apparatus cause the one or more computing apparatus to carry out the steps according to claim 1.

15. An apparatus for monitoring and/or controlling a chemical and/or biological production process of an output product, for selecting a chemical and/or biological production facility or process and/or for selecting a data driven model, the apparatus comprising processing units with instructions for monitoring and/or controlling a chemical and/or biological production process of an output product, for selecting a chemical and/or biological production facility or process and/or for selecting a data driven model, which when executed by one or more computing apparatus(es) cause the one or more computing apparatus(es) to carry out the steps according to claim 1.

16. The method of claim 6, wherein the historical data includes spectral measurement data provided from different measurement equipment of one measurement type.
